(19) United States
(12) Patent Application Publication
(10) Pub. No.: US 2019/0150754 A1
(43) Pub. Date: May 23, 2019

(54) CIRCULATORY SYSTEM MONITOR

(71) Applicant: Honeywell International Inc., Morris Plains, NJ (US)

(72) Inventors: Dinesh Naik, Bangalore (IN); Vishal Shalitkumar Kusanale, Kurundwad (IN); Ramkrishna Uchanski Pal, Bangalore (IN); Palani Thanigachalam, Bangalore (IN); Todd Eckhardt, Westerville, OH (US)

(21) Appl. No.: 15/817,008

(22) Filed: Nov. 17, 2017

(51) Int. Cl. A61B 5/022; A61B 5/021; A61B 5/00

(52) U.S. Cl. CPC A61B 5/022; A61B 2560/0223; A61B 5/0004; A61B 5/02108

(57) ABSTRACT

A circulatory system monitor may include a sensor array having a plurality of sensor assemblies and a controller in communication with the sensor array. The sensor array may be configured to be pressed against a patient's body and the controller may identify a sensor assembly of the plurality of sensor assemblies from which a signal is to be used to determine a circulatory system parameter of the patient's body. The controller may use a signal from the identified sensor assembly to determine the circulatory system parameter. The circulatory system monitor may include an inflatable member and a pump. The configuration of the sensor array and the inflatable member may facilitate ensuring alignment of at last one sensor assembly of the sensor array with a target artery to be monitored. The sensor assemblies may include a membrane that

### TECHNICAL FIELD

The present disclosure relates generally to monitoring systems, and more particularly, to monitoring systems including sensors for sensing a force or pressure applied to the sensors.

### BACKGROUND

Force sensors are often used to sense an external force applied to the sensors and provide an output signal representative of the applied force. Such sensors can be used in a wide variety of applications, including medical applications.

### SUMMARY

The present disclosure relates generally to monitoring systems, and more particularly, to monitoring systems that include force sensors for sensing a force applied to the sensors. In one illustrative configuration, a monitoring system may be a circulatory monitoring system for monitoring and/or measuring, for example, blood pressure. The circulatory monitoring system may include a force sensor array and a controller in communication with the force sensor array. In some cases, the force sensor array is pressed against a patient's body, and the controller may be configured to identify a subset of force sensors of the plurality of force sensors from which a signal is to be used to determine a circulatory system parameter such as blood pressure.

An illustrative force sensor assembly that may be used in a monitoring system may include a substrate, a sense die secured relative to the substrate, a diaphragm, a cover secured relative to the substrate, a force transfer mechanism, and a film secured to or relative to the cover. The diaphragm may be part of the sense die and/or may be positioned relative to the sense die. The cover may have an opening that may be in registration with the diaphragm when the cover is secured relative to the substrate. The force transfer mechanism may extend at least partially into the opening of the cover and may be in contact with the diaphragm. The film, when provided, may be secured to the cover over the opening and the force transfer mechanism. The force transfer mechanism may interact with the film and the diaphragm such that force transfer mechanism transfers force applied to the film to the diaphragm.

An illustrative method of using a force sensor assembly may include applying a contact force to a force sensor array having a plurality of force sensors against a target area of a patient. Each force sensor of the force sensor array may be configured to provide a signal indicative of a corresponding force applied to a respective force sensor. While the contact force is applied to the force sensor array against the target area of a patient, a circulatory system parameter may be measured based on the signals from each force sensor in the force sensor array. The method may include determining which force sensor of the force sensor array is providing a strongest measure of the circulatory system parameter (e.g. blood pressure) and use a signal from the force sensor of the force sensor array that is determined to be providing the strongest measures of the circulatory system parameter during subsequent measures of the circulatory system parameter at the target area.

The preceding summary is provided to facilitate an understanding of some of the innovative features unique to the present disclosure and is not intended to be a full description. A full appreciation of the disclosure can be gained by taking the entire specification, claims, drawings, and abstract as a whole.

### BRIEF DESCRIPTION

The disclosure may be more completely understood in consideration of the following description of various illustrative embodiments of the disclosure in connection with the accompanying drawings, in which:

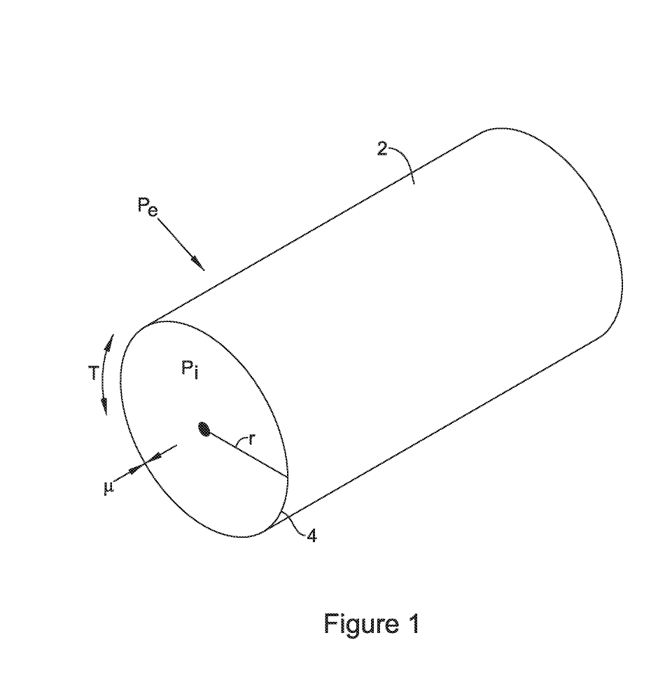

FIG. 1 is a schematic perspective view of an artery depicting elements of Laplace's Law of Cylindrical Tubes;

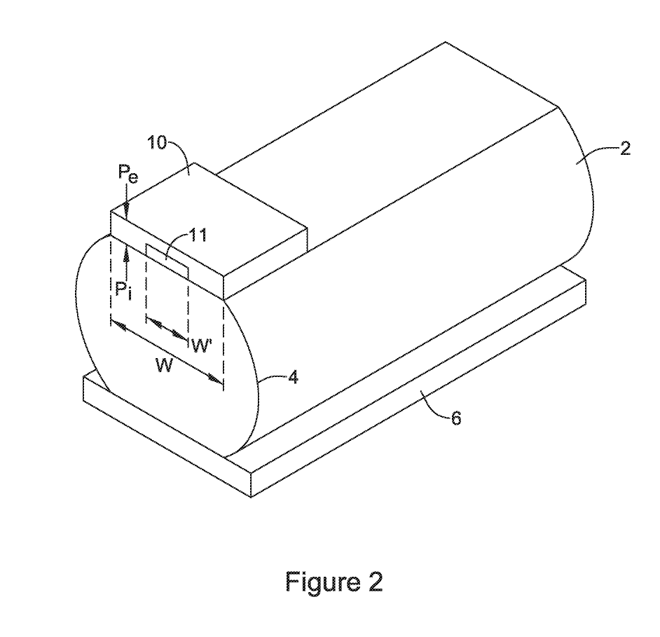

FIG. 2 is a schematic perspective view of an artery depicting Tonometry principles based on Laplace's Law of Cylindrical Tubes;

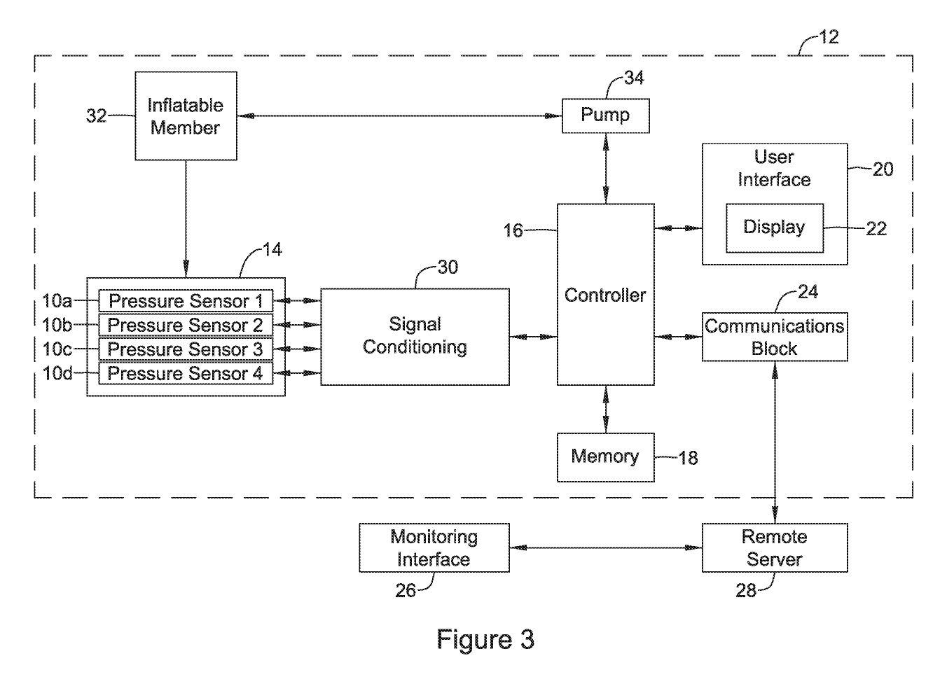

FIG. 3 is a schematic block diagram of an illustrative circulatory system monitor;

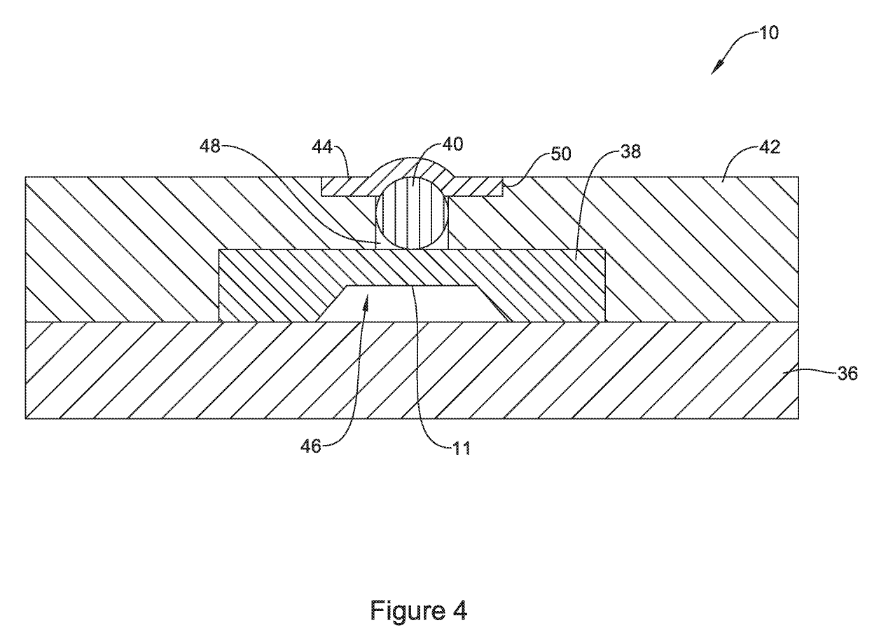

FIG. 4 is a schematic cross-sectional view of an illustrative sensor assembly with a ball-shaped force transmitting member;

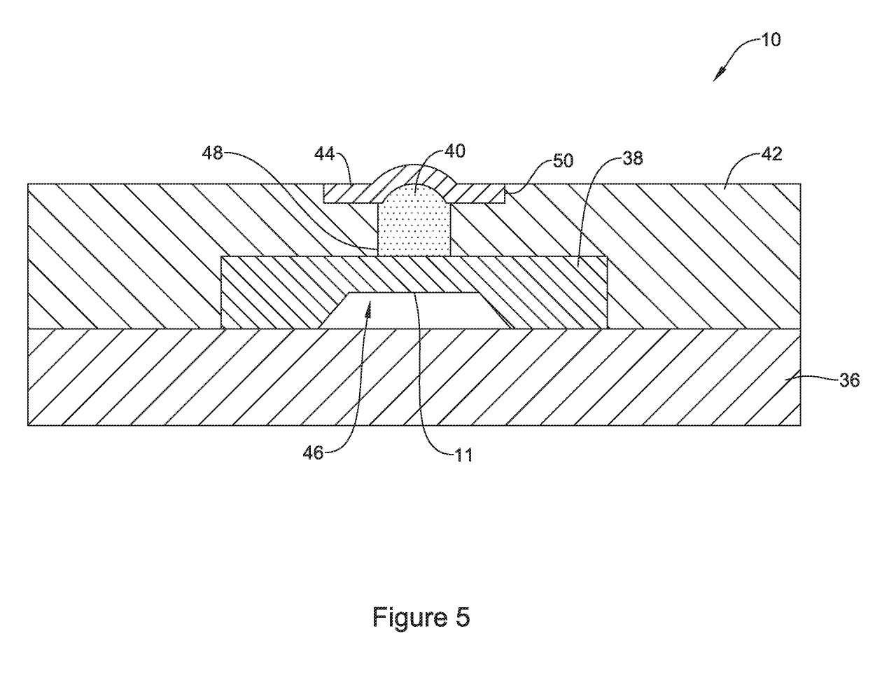

FIG. 5 is a schematic cross-sectional view of an illustrative sensor assembly with a force transmitting member formed from a fluid, gel, or semi-solid material;

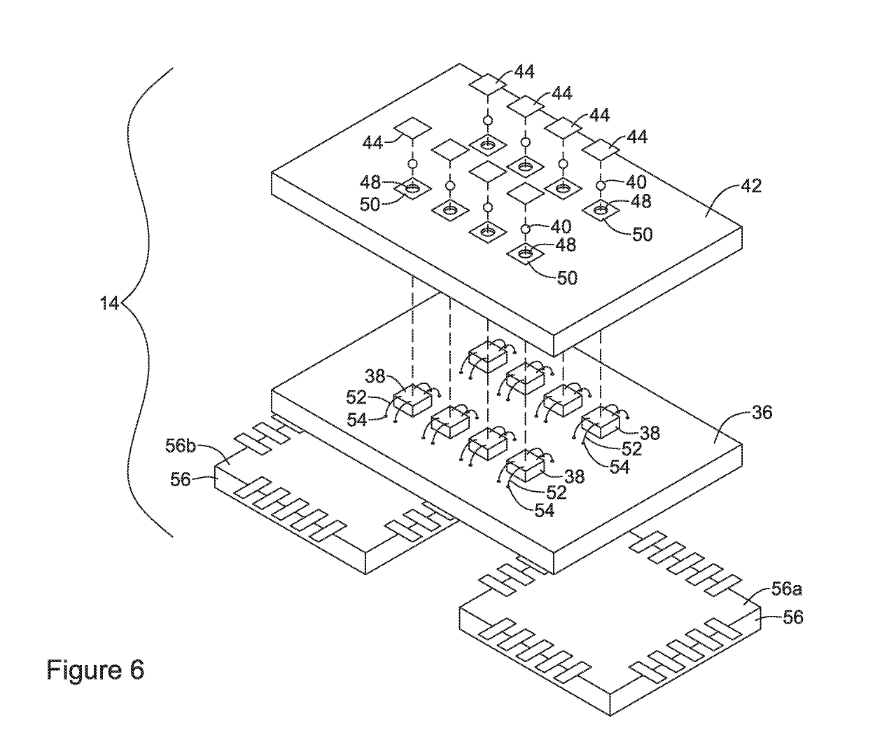

FIG. 6 is a schematic exploded perspective view of an illustrative sensor array;

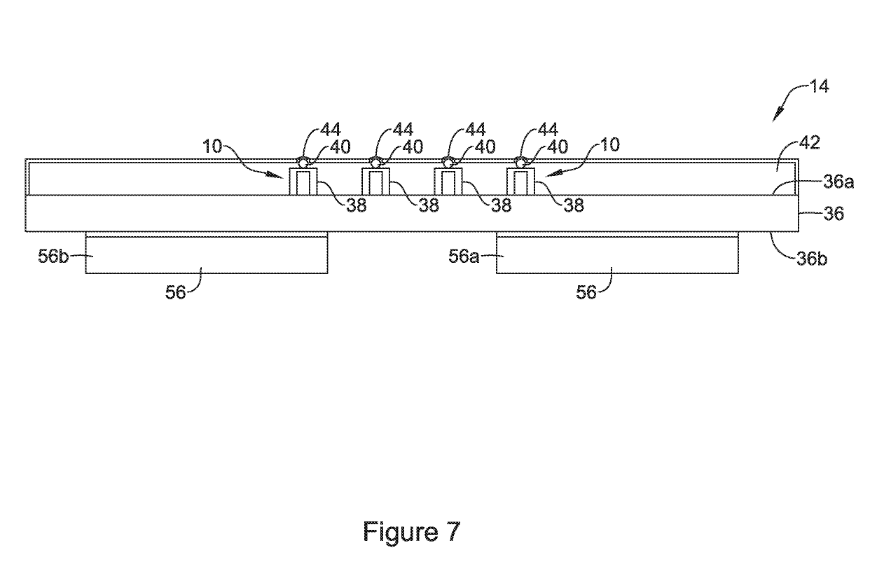

FIG. 7 is a schematic cross-sectional assembled view of the illustrative sensor array depicted in FIG. 6;

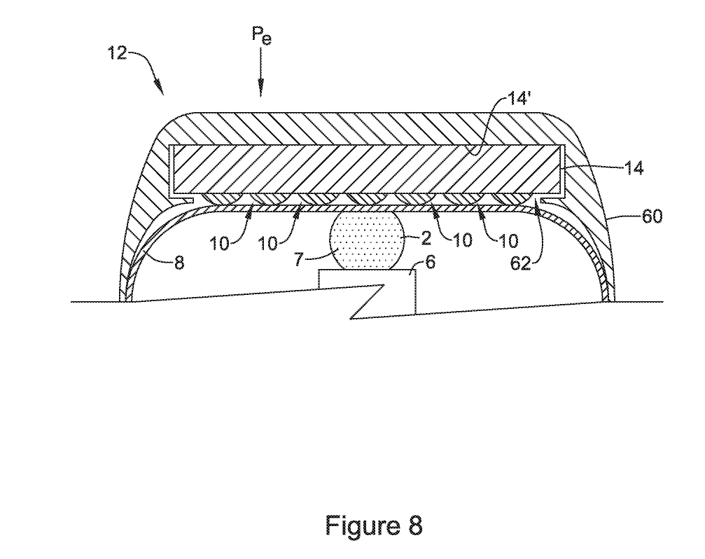

FIG. 8 is a schematic cross-sectional view of an illustrative circulatory system monitor on a patient;

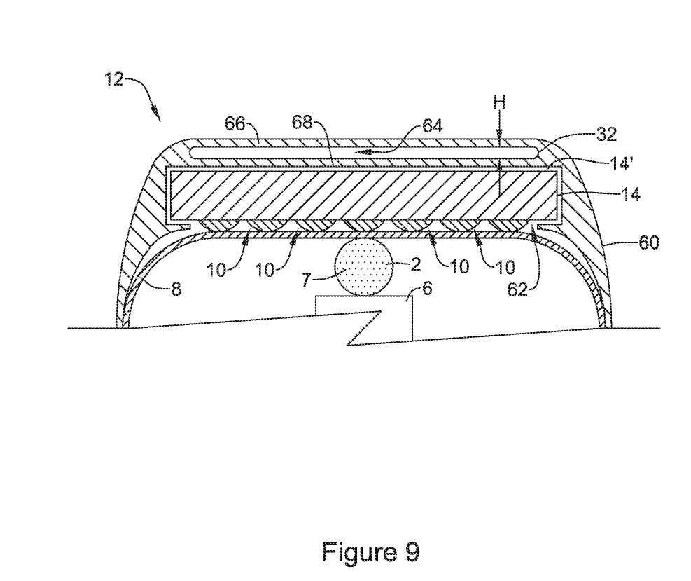

FIG. 9 is a schematic cross-sectional view of an illustrative circulatory system monitor on a patient, with an inflatable member in a deflated configuration;

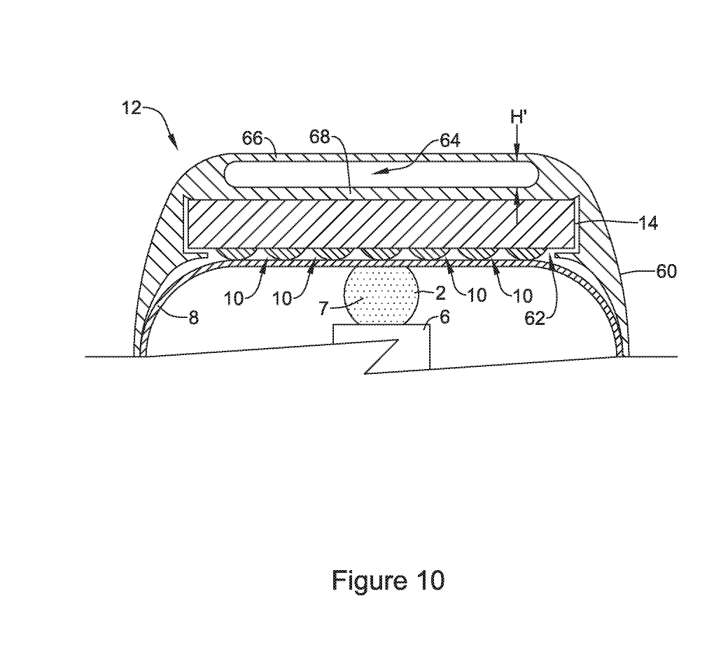

FIG. 10 is a schematic cross-sectional view of an illustrative circulatory system monitor on a patient, with an inflatable member in an inflated configuration;

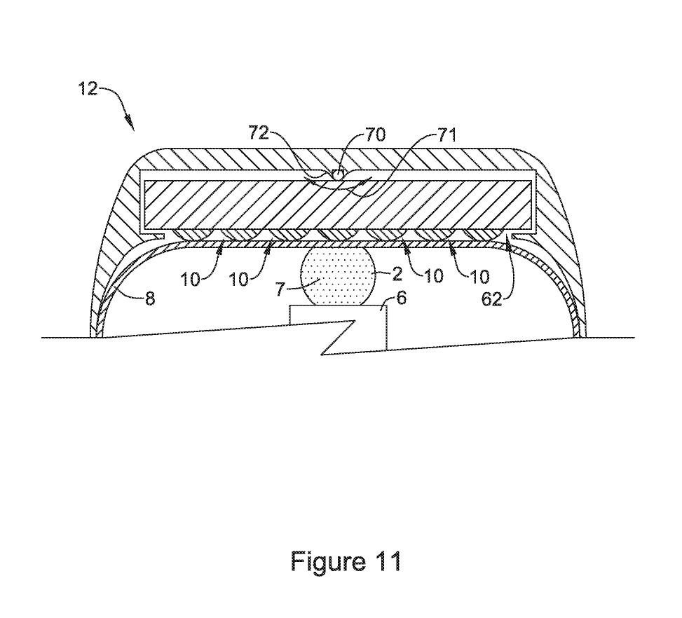

FIG. 11 is a schematic cross-sectional view of an illustrative circulatory system monitor on a patient, with a pivot element located between a sensor array and a band; and

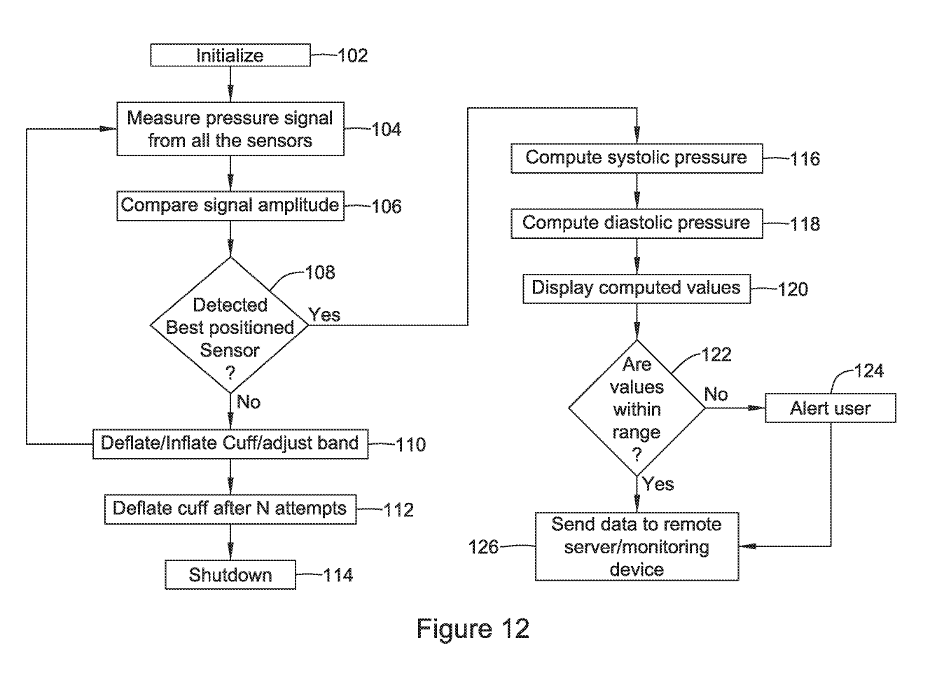

FIG. 12 is a schematic flow diagram of an illustrative calibration and computing approach for use with a circulatory system monitor.

While the disclosure is amenable to various modifications and alternative forms, specifics thereof have been shown by way of example in the drawings and will be described in detail. It should be understood, however, that the intention is not to limit aspects of the disclosure to the particular illustrative embodiments described herein. On the contrary, the intention is to cover all modifications, equivalents, and alternatives falling within the spirit and scope of the disclosure.

### DESCRIPTION

The following description should be read with reference to the drawings wherein like reference numerals indicate like elements throughout the several views. The description and drawings show several embodiments which are meant to be illustrative of the disclosure.

Blood pressure is a fundamental measurement taken in healthcare. The monitoring of blood pressure may have strong applications in many clinical fields including stroke prevention, obstetrics, neurology, vascular medicine, diabetes, and so on. Invasive measurements from an arterial line (e.g., invasive arterial blood pressure (IAP)) is generally considered to be the gold standard for blood pressure measuring. Non-invasive blood pressure (NBP) monitoring, however, may be a simpler measurement with limited, if any, patient discomfort.

The present disclosure pertains generally to sensing and monitoring circulatory system parameters, such as blood pressure, pulse, pulse wave velocity, and/or other circulatory system parameters. Although not required, the devices and methods of this disclosure may utilize or may be configured to utilize arterial tonometry to sense and monitor one or more circulatory system parameters. In some cases, the concepts of the present disclosure may be utilized for sensing and/or monitoring pressures or forces in other medical applications or non-medical applications. As such, although the disclosed concepts are described herein with respect to sensing and monitoring certain circulatory system parameters, these concepts may be applied to other medical and non-medical applications.

Arterial tonometry is a technique that may permit NBP monitoring within a superficial artery. An example superficial artery may be a radial artery of the wrist or other superficial artery of the circulatory system. The arterial tonometry technique is a way to detect blood pressure percutaneously by pressing a pressure/force sensor on a superficial artery (e.g., the radial artery near a person's wrist or other artery). In the arterial tonometry technique, part of the artery may be made flat or flatter (e.g., may be applanated) by applying a force to the skin adjacent the artery. A pressure/force sensor pressed against the skin may be used to sense the pressure/force exerted by the patient's blood against the inside of the artery wall, through the skin, and to the pressure/force sensor.

FIGS. 1 and 2 depict the physics of arterial tonometry. FIG. 1 depicts an artery 2 having an external pressure, Pₑ, applied thereto and an internal pressure, Pᵢ. The artery 2 may have a wall 4 with a wall-thickness, μ, a radius of wall curvature, r, and a wall tension, T. Laplace's Law for a cylindrical tubes states:

T=(Pₜ*r)/μ (1)

and

P=Pᵢ−Pₑ (2)

Laplace's Law of Cylindrical Tube shows a relationship between wall tension, T, and transmural pressure, Pₜ, and the radius of wall curvature, r. Based on this relationship, when a sensor assembly 10 applanates the wall 4 of the artery 2, as shown for example in FIG. 2, the radius of the wall curvature, r, goes to infinity and external pressure, Pₑ, acting on the sensor assembly 10 to sandwich the artery 2 between the sensor assembly 10 and a bone, muscle or other anatomical structure 6 is equivalent to internal pressure, P within the artery 2.

$${P}_{t} = Pi - Pe = \frac{μ T}{r} r \to ∞ = 0 \qquad ( 3 )$$

As a result, the pressure acting on the sensor assembly 10 corresponds to the internal pressure and an output from the sensor assembly 10 reflects the internal pressure, Pᵢ, of the artery 2 and thus, arterial tonometry techniques may be utilized for sensing blood pressure and/or other related parameters of the circulatory system in a non-invasive manner (e.g., from exterior the skin of a patient or at least exterior the vessel in which a pressure or other parameter is being monitored). For best measurements of pressure within the artery 2 using arterial tonometry, a diaphragm 11 of the sensor assembly 10 may have a width, W′, that is less than a width, W, of the surface of the artery 2 that is applanated by the sensor assembly 10.

Arterial tonometry may be sensitive to alignment of a diaphragm of the sensor assembly 10 with the flatter part (e.g., an applanated part) of the artery. The present disclosure discloses a circulatory system monitor 12 that may help address this sensitivity by facilitating optimal placement of one or more sensor assemblies 10 relative to the applanated part of the artery to obtain strong and accurate pressure/force measurements. For example, the disclosed circulatory system monitor 12 may facilitate placing a pressure sensor assembly 10 entirely or substantially entirely within the width, W, of the applanated surface of the artery.

A circulatory system monitor 12 may take on any suitable form. In some cases, the circulatory system monitor 12 may have a closed loop configuration. FIG. 3 depicts a schematic diagram of an illustrative closed loop circulatory system monitor 12. Although the circulatory system monitor 12 is depicted as having a closed loop, such a closed loop configuration is not required in all cases.

As shown in FIG. 3, the circulatory system monitor 12 may include a sensor array 14 having a plurality of sensor assemblies 10 (e.g., pressure and/or force sensor assemblies) and a controller 16 in communication with the sensor array 14. The controller 16 may be configured to receive signals from the sensor assemblies 10 of the sensor array 14 and identify one (or more) of the sensor assemblies 10 from which a signal is to be used in current and/or future analyses, and then determine a circulatory system parameter based on the signal from the identified sensor assembly 10. In some cases, the controller 16 may determine the circulatory system parameter based only on the signal from the identified sensor assembly 10 and/or the controller 16 may determine the circulatory system parameter based on signals from a plurality of sensor assemblies 10 including or excluding signals from the identified sensor assembly 10. As is discussed in greater detail below, the identified sensor assembly 10 may be identified by the controller 16 in one or more manners including, but not limited to, by identifying the sensor assembly 10 with the strongest signal, most consistent signal, signal closest to an expected signal range, and/or one or more other signal related parameters. Circulatory system parameters sensed or determined by the controller 16 may include, but are not limited, to blood pressure, pulse rate, pulse wave velocity, etc.

In addition to performing analyses on signals received from the sensor array 14, the controller 16 may be configured to control operation of the sensor array 14. In one example, the controller 16 may be configured to turn on and/or off various sensor assemblies 10 of the sensor array 14 to limit noise and/or reduce power consumed by the sensor array 14 after one or more sensors have been identified for use in taking measurements. As shown for example in FIG. 3, the sensor array 14 may include a first sensor assembly 10a, a second sensor assembly 10b, a third sensor assembly 10c, and a fourth sensory assembly 10d. In such instances and when the controller 16 is monitoring blood pressure of a patient, the controller 16 may identify which sensor assembly 10 is best positioned with respect to the patient's artery based on signals from the sensor assemblies 10a, 10b, 10c, 10d, and then turn off and/or limit power supplied to one or more of the sensor assemblies 10 not identified as being in the best position for sensing the patient's blood pressure.

The controller 16 may include a microprocessor or microcontroller or the like, and may be operatively coupled to a memory 18. The memory 18 may be used to store any desired information, such as control algorithms, set points, schedule times, diagnostic limits such as, for example, blood pressure limits, pulse rate limits, pulse wave form limits, and the like. The memory 18 may be any suitable type of storage device including, but not limited to, RAM, ROM, EPROM, flash memory, a hard drive, and/or the like. In some cases, the controller 16 may store information within the memory 18, and may subsequently retrieve the stored information from the memory 18. In some cases, the controller 16 may include and/or be in communication with a timer (not shown).

The illustrative circulatory system monitor 12 may optionally include signal conditioning circuitry 30, as shown in FIG. 3. In instances where signal condition circuitry 30 is included in circulatory system monitor 12, the signal condition circuitry 30 may be mounted on a substrate (e.g., as discussed with respect to FIGS. 5 and 6). In some cases, the signal conditioning circuitry 30 may include a microprocessor and/or a microcontroller, an application specific integrated circuit (“ASIC”), and/or an application specific standard product (“ASSP”). In some cases, the signal conditioning circuitry 30 may be (at least partially) incorporated into the controller 16.

When provided, the signal conditioning circuitry 30 may include circuitry that receives an output signal from the sensor array 14, and in response may generate output signals having magnitudes that are representative of a magnitude of a force or pressure applied to the sensor assemblies 10 of the sensor array 14. The signal conditioning circuitry 30 may condition the output signals of the sensor assemblies 10 to correct for repeatable variations, such as offset, sensitivity, non-linearity, temperature effects, and/or other variations. Further, the signal conditioning circuitry 30 may condition the output signal to compensate for temperature-dependent variations in the electrical characteristics and/or to account for a nonlinear relationship between changes in the electrical characteristic and corresponding changes in the magnitude of the force. In some cases, the signal conditioning circuitry 30 may facilitate comparing measurement signals to reference signals from the sensor assemblies 10 of the sensor array 14.

The circulatory system monitor 12 may include a user interface 20 operatively coupled to the controller 16, but this is not required, where the user interface 20 may include a display 22 in communication with the controller 16 to display information related to monitored circulatory system parameters. The user interface 20, when provided, may be any suitable user interface that permits the circulatory system monitor 12 to display and/or solicit information, as well as accept one or more user interactions with the circulatory system monitor 12. For example, the user interface 20 may permit a user to locally enter data such as diagnostic limits, configuration settings, response configurations for responding to alerts, and the like. The user interface 20 may take the form of a watch face-like user interface or may be a user interface on a device remote from the sensor array 14.

In one example, the user interface 20 may be a physical user interface that is accessible at the circulatory system monitor 12, and may include a display 22 and/or a distinct keypad. The display 22 may be any suitable display and in some cases may be backlit or have a different lighting configuration. In some instances, a display may include or may be a liquid crystal display (LCD), and in some cases an e-ink display, fixed segment display, a light emitting diode (LED) display, or a dot matrix LCD display. Alternatively or in addition, the user interface 20 may be a touch screen LCD and/or LED panel or other touch sensitive screen that functions as both display and keypad. The touch screen panel may be adapted to solicit values and/or selections for a number of operating parameters and/or to receive such values, but this is not required. In still other cases, the user interface 20 may be a dynamic graphical user interface.

The circulatory system monitor 12 may be a connected device that is capable of connecting to one or more networks (e.g., wired and/or wireless local area network (LAN), wide area network or global network (WAN) including, for example, the Internet). In some cases, the circulatory system monitor 12 may be accessed from, controlled from, and/or have data therefrom analyzed at a remote location over the network using a remote wired or wireless device such as, for example, a smart phone, a tablet computer, a laptop or personal computer, a wireless network-enabled key fob, an e-reader, a remote server, and/or the like.

To facilitate connecting to other devices, the circulatory system monitor 12 may include a communications block 24 (e.g., a communications component) in communication with the controller 16 and having one or more communication ports. The communications block 24 may facilitate connecting to other devices via a wired or wireless communication link. In one example, the communications block 24 may facilitate communicating with and/or connecting to other devices via one or more communication protocols including, but not limited to, cellular communication, ZigBee, Bluetooth, WiFi, IrDA, dedicated short range communication (DSRC), EnOcean, and/or any other suitable common or proprietary wireless protocol, as desired.

The circulatory system monitor 12 may be in communication with a remote computing device(s) (e.g., a monitoring interface 26, remote server 28, and/or other device) via the communications block 24 and communicate information (e.g., data, analyses, alerts, etc.) from the controller 16 to the remote computing device(s). In some cases, the circulatory system monitor 12 may communicate directly to the remote server 28. The remote server 28 may analyze information and/or data received from the circulatory system monitor 12 and send results and/or alerts back to the circulatory system monitor 12 or to the monitoring interface 26. Alternatively or in addition, when an alert is identified, the remote server 28 may send the alert to the monitoring interface 26 and/or the circulatory system monitor 12. The circulatory system monitor 12 may display the alert on the user interface 20 of the circulatory system monitor 12.

The monitoring interface 26 may include any suitable computing device. In one example, the monitoring interface 26 may be a remote or local user device including, but not limited to a smart phone, a tablet computer, a laptop or personal computer, a wireless network-enabled key fob, an e-reader, and/or the like.

To facilitate applanating of the patient's artery with the sensor array 14, the circulatory system monitor 12 may include, for example, an inflatable member 32 and a pump 34 in communication with the inflatable member 32 for inflating and/or deflating the inflatable member 32, The inflatable member 32, when inflated, may provide an inward force to the sensor array 14 and against the artery. The inflatable member 32 may be part of a strap, band or other mechanism that extends around the patient's wrist or the like, with the sensor array 14 between the inflatable member 32 and the patient's skin. Alternatively or in addition, the circulatory system monitor 12 may include one or more other mechanisms for applanating a patient's artery including, but not limited to, adjustable straps and/or other manual or automated adjustable mechanisms configured to provide a radially inward force on a portion of a patient's skin.

When provided, the inflatable member 32 may be in mechanical communication with the sensor array 14 and may be configured to apply a force to the sensor array 14 against the patient's body. In some cases, the inflatable member 32 may be in direct contact with the sensor array 14 (e.g., the inflatable member 32 may contact a portion of the sensor array 14) or indirect contact with the sensor array 14 (e.g., the inflatable member 32 may interact with the sensor array 14 through a membrane or the like, by causing a tightness of a band including the sensor array 14 to change based on inflating or deflating the inflatable member 32).

A pump 34 may be any suitable type of pump. The pump 34 may be a manual operated pump, or may include a motor or the like to operate the pump. The pump 34 may be connected to the inflatable member 32 or configured to be selectively connected to the inflatable member 32. The pump 34 may be manually actuated or actuated in an automated manner to inflate and/or deflate the inflatable member 32. In some cases, the pump 34 and/or the inflatable member 32 may be connected to and/or in communication with the controller 16 to monitor an amount of force applied to the sensor assembly 10 by the inflatable member 32 and/or to actuate the pump 34.

FIGS. 4 and 5 depict a schematic cross-sectional views of the illustrative sensor assembly 10. The illustrative sensor assembly 10 includes a substrate 36, a sense die 38 defining or including the diaphragm 11, a force transmitting member 40 (e.g., a force transfer mechanism) configured to transmit a force acting thereon to the diaphragm 11, a cover 42 extending over at least a portion of the substrate 36 and the sense die 38, and a membrane 44 extending over the force transmitting member 40 to help provide a seal over the force transmitting membrane. Additionally or alternatively, the sensor assembly 10 may include one or more other features including, but not limited to, structural supports, wire bonds, electrical traces, bond pads, one or more constraints, and/or other features facilitating sensing and/or monitoring a force or pressure.

The substrate 36 may be a suitable type of substrate configured to support the sense die 38. In some cases, the substrate 36 may be a printed circuit board (PCB), a printed wiring assembly (PWA), a printed wiring board (PWB), and/or other device configured to facilitate supporting the sense die 38 and/or other electronic components. In some cases, the substrate 36 may be a thick film printed ceramic board, but this is not required. In one example, the substrate 36 may be made, at least in part, of FR 4 laminate and/or other material. Alternatively or in addition, the substrate 36 may be a flexible PCB.

The sense die 38 of the sensor assembly 10 may be arranged to sense an absolute pressure, where a pressure or force acting on the force transmitting member 40 and the diaphragm 11 is referenced against a vacuum pressure. When sensing an absolute pressure, the sense die 38 may be fabricated to include a vacuum or reference cavity 46 immediately behind the diaphragm 11, such that a pressure or force acting on the force transmitting member 40 and/or the diaphragm 11 is referenced against a vacuum (not specifically shown) or other reference pressure. Alternatively, the sense die 38 may be arranged to sense a gauge pressure. In such a gauge pressure sensor, the substrate 36 may include an opening 48 extending from the sense die 38 through the substrate 36 (e.g., extending through the substrate 36 from a first side to a second side of the substrate 36) to allow a reference pressure (e.g. atmosphere) to reach the back side of the sense die 38. Example sensor die may include, but are not limited to, those described in U.S. Pat. Nos.: 7,503,221; 7,493,822; 7,216,547; 7,082,835; 6,923,069; 6,877,380, and U.S. patent application publications: 2010/0180688; 2010/0064818; 2010/00184324; 2007/0095144; and 2003/0167851, all of which are hereby incorporated by reference.

The cover 42 may be any suitable type of cover configured to help protect the sense die 38 from external forces and material (e.g. fluids) that may affect the sensing and/or monitoring capabilities of the sensor assembly 10. In some cases, the cover 42 may be made of metal, polymer, and/or other suitable material. In some cases, the cover 42 may include an opening 48 for receiving the force transmitting member 40, at least partially containing the force transmitting member 40, and allowing the force transmitting member 40 to contact the diaphragm 11.

The force transmitting member 40 may have any suitable configuration for transferring a force or pressure acting on the force transmitting member 40 to the diaphragm 11. In some cases, the force transmitting member 40 may include a spherical object, as shown in FIG. 4, a pin, an extender, a button, any other force transmitting device, and/or a combination thereof. If desired, only part of the outside surface of the force transmitting member 40 may be spherical in shape or take on a particular shape. The force transmitting member 40 may be made of any material. For example, the force transmitting member 40 may include stainless steel, other suitable metal and/or other suitable material. In some cases, the force transmitting member 40 may include a ball bearing (e.g., a stainless steel ball bearing or other ball bearing). It is contemplated, however, that other generally spherical and other shaped elements may be used as or as part of the force transmitting member 40, if desired, including polymer based objects.

In some cases, the force transmitting member 40 may be made from a suitable fluid, gel, or semi-solid material, as shown in FIG. 5. Example types of fluid, gel, or semi-solid materials may include dielectric material, non-compressible material, biocompatible material, colored material, non-colored material, and/or one or more other types of material. Example materials acceptable for use as or in fluid, gel, or semi-solid force transmitting members 40 may include fluoro-silicone gel, a cured silicone rubber or silicone elastomer, a cured liquid silicone rubber, an oil and/or any other suitable material. When the force transmitting member 40 is a non-compressible fluid or gel, it may be at least partially contained in the opening 48 of the cover 42 by the membrane 44.

Silicone elastomers may be polysiloxanes and/or polydimethylsiloxanes. Example silicone elastomers may include SILASTIC® MDX4-421 Biomedical grade elastomer from Dow Corning Corporation, SILPURAN® 2430 (e.g., an addition curing RTV silicone rubber curing to a silicon elastomer) from Wacker Chemie AG, and/or one or more other silicone elastomers.

The membrane 44 may extend over the force transmitting member 40 and the opening 48 to protect the force transmitting member 40 and the sense die 38 from contaminants (e.g. sweat and/or other body fluids or the like). In some cases, the membrane 44 may form a hermetic seal with the cover 42. The membrane 44 may be made from a suitable material and have a suitable thickness that allows for a force or pressure to be transferred to the force transmitting member 40 while still protecting the force transmitting member 40 and the sense die 38 from contaminants. Illustrative membranes 44 may be made from polymers, nylon, a cured silicon elastomer, and/or other materials. In some case, the material of the membrane 44 may be a film element. In one example, the membrane 44 may be a nylon film. Although a thickness of the membrane 44 may differ based on the material of the membrane 44, the membrane 44 may generally have a thickness between about 0.01 millimeter (mm) or thinner and 1.0 mm or thicker. In one example, a membrane may have a thickness between about 0.1 mm and 0.2 mm. The membrane 44 may be connected to the cover 42 in any suitable manner. In some cases, the membrane 44 may be secured to the cover 42 with an adhesive, a welding technique, a laser welding technique, a molding technique, and/or other suitable techniques.

To facilitate a connection between the membrane 44 and the cover 42, the cover 42 may have a recess or an indentation 50 extending entirely or at least partially around a perimeter of the opening 48 for receiving the membrane 44, but this is not required. When included, the indentation 50 may have a depth equal to or substantially equal to a thickness of the membrane 44 or other depth.

In some cases, the membrane 44 and the cover 42 may interact to render the sensor assembly 10 compliant with at least an ingress protection (IP) 67 rating, as defined by international standard 60529 of the International Electrotechnical Commission (i.e., IEC 60529).

The first digit in an IP 67 rating (i.e., the “6”) indicates such a sensor assembly 10 is dust tight (e.g., there is no ingress of dust to the sense die 38 and there is complete protection against contact). The second digit in an IP 67 rating (i.e., the “7”) indicates such a sensor assembly 10 may be immersed in up to one (1) meter (m) of water without harm to the sense die 38. One example membrane 44 and cover 42 that would receive the IP 67 rating may be a nylon membrane 44 having a thickness between about 0.1 mm and about 0.2 mm, where the membrane 44 is laser welded to the cover 42 around an entirety or substantially an entirety of a perimeter of the opening 48 in the cover 42.

FIG. 6 is a perspective exploded view of a packaging of the sensor array 14, including the substrate 36, a plurality of sense die 38 (e.g., two rows of four sense die 38 in each row or any other suitable sense die 38 configuration), the cover 42, a plurality of force transmitting members 40 configured to be received within corresponding openings 48 of the cover 42, and a plurality of membranes 44 each configured to be received within a corresponding recess or indentation 50 in the cover 42. Alternatively, the sensor array 14 may take on one or more other configurations. In one example of a different configuration, one or more of the sense die 38 may have its own cover 42 and/or own substrate 36 separate from a cover 42 and/or substrate 36, respectively, of one of the other sense die 38. A layout of the plurality of sense die 38 may have any suitable configuration and may be configured to position at least one sense die 38 directly over an artery or other vessel of the circulatory system for monitoring, and at least one other sense die 38 spaced sufficiently far so as to not be directly over the same artery or other vessel of the circulatory system.

Components of the sensor array 14 may be connected to one another with one or more suitable connections including, but not limited to welding, soldering, adhesives, and/or one or more other connections. In some cases, the components of the sensor array 14 may be connected to one another using an adhesive such as a silicone, RTV, a silicone-epoxy, a soft epoxy, or a regular or hard epoxy. The adhesive may include a conductive adhesive, a nonconductive adhesive, or a combination of conductive and nonconductive adhesives. Any suitable conductive adhesive and nonconductive adhesive may be used. One example nonconductive adhesive is RTV6424, which is available from Momentive Performance Materials Inc. of Waterford, N.Y. One example conductive adhesive may be SDC5000, which is available from Momentive Performance Materials Inc. of Waterford, N.Y. These are just examples, and it is contemplated that any other suitable conductive and/or nonconductive adhesive may be used, as desired. When provided, the combination of conductive and nonconductive adhesives may be provided in a pattern to electrically connect electronic components of the sensor array 14.

The sense die 38 may be connected to the substrate 36 in any suitable manner such that sense die 38 provides electrical signals to electrically conducive components in the substrate 36. In one example, as shown in FIG. 6, the sense die 38 may be electrically connected to bond pads 54 on the substrate 36 via wire bonds 52.

In some cases, the substrate 36 may also include signal conditioning circuitry 30 that incorporates one or more ASSPs or ASICs 56. The one or more ASSPs or ASICs 56 may be attached to a same side of the substrate 36 to which the sense dies 38 are attached and/or to an opposite of the substrate 36 to which the sense dies 38 are attached. In some cases, and as shown in FIG. 6, the sensor array 14 may include a first ASIC 56a and a second ASIC 56b configured to condition signals received from the sense die 38. The ASICs 56a, 56b are shown electrically connected to the back side of the substrate 36 via wire bonds, bump bonds, electrical terminals, and/or any other suitable electrical connections. Although not particularly shown in

FIG. 6, the controller 16, the communications block 24, and/or other electronic components may be on and/or electrically connect to the substrate 36, as desired.

The sensor assemblies 10 of the sensor array 14 may be split into at least a first set of sensor assemblies 10 (e.g., a first row of sensor assemblies 10) and a second set of sensor assemblies 10 (e.g., a second row of sensor assemblies 10). The first set of sensor assemblies 10 may be configured to output signals to the ASICs 56 (e.g., to the first ASIC 56a) and the second set of sensor assemblies 10 may be configured to output signals to the ASICs 56 (e.g., to the second ASIC 56b). In some cases, the controller 16 may compare an output of certain sensor assemblies 10 with the output of certain other sensor assemblies 10 to help identify which sensor assemblies 10 to use when determining certain circulatory system parameters.

In some instances, the ASICs 56 and/or other circuitry of the substrate 36 may include circuitry that is configured to format one or more output signals provided by the sensor assemblies 10 into a particular output format. For example, circuitry of the ASICs 56 and/or the substrate 36 may be configured to format the output signal provided by sensor assemblies 10 into a ratio-metric output format, a current format, a digital output format and/or any other suitable format. In some cases, the ASICs 56 and/or other circuitry of the substrate 36 may be configured to regulate an output voltage. The ASICs 56 and/or other circuitry on the substrate 36 may include traces and/or other circuitry that may serve as a conduit to test pads, and/or for providing ratio-metric (or other) output to one or more electrical terminals facilitating electrical connections with electronic components of one or more devices used with the sensor array 14.

FIG. 7 is a schematic cross-sectional view of the packaging of the sensor array 14 taken along a row of sensor assemblies 10. In the example of FIG. 7, the sense dies 38 and the cover 42 are attached to a first side 36a of the substrate 36 and the ASICs 56a, 56b are attached to a second side 36b of the substrate 36. Further, the force transmitting members 40 are received within apertures of the cover 42 and may cause the membrane 44 to protrude out from an outer surface of cover 42. Such protrusion may facilitate interaction with a pressure or force exterior (e.g. artery force) to the corresponding sensor of the sensor array 14.

FIGS. 8-11 depict a schematic cross-sectional views of the circulatory system monitor 12 with the sensor array 14 incorporated into a band 60 (e.g., a wrist band, head band, arm band, watch band, cuff, an ankle band, a neck band, etc.) extending around a suitable portion of a patient's body (e.g., wrist, arm, head, ankle, leg, neck, etc.) for monitoring circulatory system parameters. When the sensor array 14 is incorporated into the band 60, the user interface 20 may be a watch face-like user interface in the band 60 and/or the user interface 20 may be separate from the band 60 (e.g. on the user's smartphone or the like). Although the circulatory system monitor 12 is depicted in FIGS. 7-10 with the sensor array 14 being incorporated into the band 60, the circulatory system monitor 12 may take on one or more other configurations suitable for being used to monitor circulatory system parameters. Whether having a band configuration or other configuration, it is contemplated that the circulatory system monitor 12 may be incorporated into a garment or the like that can be worn by a patient (e.g., a shirt, pants, a belt, or the like), but this is not required.

In the example shown, the band 60 may be configured to extend around a portion of a patient's body. Although not particularly shown in the Figures, the band 60 may be a continuous single strap, a strap having two ends, a stretchable strap, an elastic strap, and/or other strap configuration. In some cases, ends of the band 60 may be connectable with a buckle, hook and loop fasteners, magnets, knots, and/or other connector. The band 60 may be manually tightened or loosened around a patient and/or tightened or loosened in an automated manner (e.g. inflatable member 32).

The band 60 may have an opening 62 for receiving the sensor array 14. In some cases, the opening 62 in the band 60 may be sized to have a volume greater than the volume of the sensor array 14 to allow for adjustment of the sensor array 14 within the opening 62 as the band 60 is tightened around the portion of the patient. Such potential for adjustment of the sensor array 14 relative to the band 60 may facilitate one or more of the sensor assemblies 10 being in good contact with an artery 2 of the patient having blood 7 passing therethrough. In other cases, the band or cuff may be moved relative to the patient's body to facilitate one or more of the sensor assemblies 10 being in good contact with an artery 2 of the patient having blood 7 passing therethrough.

The sensor array 14 may be positioned within the opening 62 of the band 60 such that the sensor assemblies 10 may extend out of the opening 62 in a manner configured to interact with a patient's skin 8. When the band 60 has been tightened around a portion of the patient, a wall of the opening 62 is configured to act on a top side 14′ of the sensor array 14 and cause the sensor assembly 10 to apply an exterior force or pressure, Pₑ, to the patient's skin 8 and the patient's artery 2 (e.g., a radial artery or other artery) resulting in applanation of the artery 2, as shown in FIG. 8. In some cases, the application of the exterior force or pressure, Pₑ, to the skin may result in applanating a surface of the artery 2 against the skin 8 and against the bone (e.g., a radial bone), muscle or other anatomical structure 6, as shown in FIG. 8. Once, the band is properly tightened around a portion of a patient, the sensor assemblies 10 may begin sensing circulatory system parameters.

FIGS. 9 and 10 depict the circulatory system monitor 12 with the sensor array 14 within an opening 62 of a band 60 having an inflatable member 32. The inflatable member 32 may be configured to inflate in order to apply an inward pressure against the sensor array 14 to applanate the artery 2, as shown for example in FIG. 10, and deflate to remove pressure applied by the circulatory system monitor 12 and allow the artery 2 to take its natural form due to blood 7 passing there through, as shown for example in FIG. 9. As discussed above, but not shown in FIGS. 8 and 9, the inflatable member 32 may be in communication with a pump (manual or motor driven) to allow the pump to inflate the inflatable member 32 to an inflated configuration having a height, H′, and/or deflate the inflatable member 32 to a deflated configuration having a height, H. The pump may be incorporated into the band 60 or may be separate from the band 60. In some cases, the inflatable member 32 may be in communication with an exhaust valve (not shown) that facilitates releasing fluid (e.g. air) from the inflatable member 32 to deflate the inflatable member 32 to the deflated configuration seen in FIG. 9. The inflatable member 32 may be formed from a cavity 64 in the band 60 or may be a separate inflatable component that is positioned within a cavity 64 in the band 60. In some cases, the inflatable member 32 may have and/or may abut a more stiff side and a less stiff side of the cavity 64, where the less stiff side may form a side of the opening 62 in the band 60 and may act on the sensor array 14 when the inflatable member 32 is inflated. As shown in FIGS. 8 and 9, the inflatable member 32 may be formed by cavity 64 in the band 60, where the cavity 64 is defined by at least a first side 66 (e.g., a more stiff side) and a second side 68 (e.g., a less stiff side) that is configured to flex more than the first side 66 when the inflatable member 32 is inflated or in an inflated configuration.

FIG. 11 depicts a cross-sectional view of the circulatory system monitor 12 having a sensor array 14 within a band 60 configuration around a portion of a patient and applanating the artery 2 of the patient. FIG. 11 depicts a pivot element 70 in contact with a surface of the sensor array 14 that is opposed to a surface of the circulatory system monitor 12 from which the sensor assemblies 10 extend. The pivot element 70 also engages or extend from a wall of the opening 62 in the band 60 and may facilitate movement of the sensor array 14 within the opening 62 of the band 60. Utilization of a pivot element (e.g., the pivot element 70) may allow an orientation of the sensor array 14 within the opening 62 to adjust, at least partially, about the pivot element 70 in a direction of arrows 71 (and in some cases other directions) to allow the sensor assemblies 10 to make good contact with the patient's skin even when the band 60 is not itself perfectly aligned with the patient's skin.

The pivot element may be any suitable element for allowing the sensor array 14 to orient itself relative to the patient's skin. In some cases, the pivot element 70 may include a rounded portion or may be a spherical ball about which the sensor array 14 may be able to pivot. Alternatively or in addition, the pivot element 70 may be an extension or protrusion of the band 60 about which the sensor array 14 may pivot. When the pivot element 70 is at least partially separate from the band 60, the pivot element 70 may be secured within a pocket 72 of the band 60. In some cases, the pivot element 70 may be snap fitted into the pocket 72. Alternatively or in addition, the pivot element 70 may be rigidly secured to the band 60 such that the pivot element 70 cannot be naturally separated from the band 60. While the band shown in FIG. 11 does not explicitly show an inflatable member 32, it is contemplated that the pivot element 70 may be used in conjunction with an inflatable member 32.

The illustrative circulatory system monitor 12 described herein may use a calibration and computing approach 100, as shown in FIG. 12 for example, to automatically detect which sensor assembly 10 (e.g., sensor assembly 10 or other sensor assembly) of a sensor array (e.g., the sensor array 14 or other sensor array) is in good contact and aligned with an artery or other target vessel of the circulatory system. The first step of the calibration and computing approach 100 may be to initialize 102 the circulatory system monitor 12 on a portion of a patient (e.g., on an arm, ankle, neck, head, wrist, etc. of a patient). When the circulatory system monitor 12 includes a band (e.g., the band 60 or other band), initializing 102 may entail, among other steps, placing the band around the portion of the patient and adjusting the circulatory system monitor 12 to a desired fit around the portion of the patient, such as by adjusting a length of the band, inflating/deflating an inflatable member 32, etc.). The desired fit may apply a contact force against a target area of a patient (e.g., adjacent an artery or other vessel) to the sensor array 14 having a plurality of sensor assemblies 10, wherein each sensor assembly 10 of the sensor array 14 may be configured to provide a signal indicative of a corresponding force. In some cases, initializing the circulatory system monitor 12 may also include booting up or turning on the electronic components of the circulatory system monitor 12.

Once the circulatory system monitor 12 has been initialized, a circulatory system parameter may be measured based on the signals from each sensor assembly 10 in the sensor array 14 while the contact force is applied to the sensor array 14 against the target area of a patient. For example, signals from all or substantially all of the sensor assemblies 10 of the sensor array 14 may be measured at 104, and the amplitudes of the signals from each indicative of a circulatory system parameter may be compared 106 to determine which sensor assembly 10 is providing the best or strongest signal.

Once the measures of the signals have been compared, the circulatory system monitor 12 may determine which sensor assembly 10 of the sensor array 14 is providing a best signal and/or strongest measure of the circulatory system parameter, as shown at 108. The best signal may be based on one or more factors in addition to or as an alternative to the greatest signal amplitude including, but not limited to, most consistent signal, a signal closest to an expected signal, a signal with the highest signal-to-noise ratio, and/or one or more other signal related factors. This may identify a best positioned sensor relative to a target artery or other vessel for taking pressure measurements and/or other measurements within the target artery or other vessel.

If no best-positioned sensor is detected and/or no sensor signals meet a baseline requirement, the calibration and computing approach 100 may include manually and/or automatically adjusting 110 the position or tightness of the band. In some cases, adjusting the band of the circulatory system monitor 12 may include inflating an inflatable member (e.g., the inflatable member 32 or other inflatable member), deflating the inflatable member 32, adjusting straps of the band, and/or otherwise physically adjusting the circulatory system monitor 12. After the band has been adjusted 110, steps 104-110 may be repeated until a best-positioned sensor is identified at step 108.

In some cases, after N number of attempts at determining a best-positioned sensor with a best signal that meets a baseline reading without identifying the sensor with a best signal, the circulatory system monitor 12 may determine a best-positioned sensor cannot be identified at this time and deflate 112 the inflatable member 32 and shut down 114 the circulatory system monitor 12. In some case, the N number of attempts at determining a best-positioned sensor with a best signal that meets a baseline reading may be pre-set or set by a user through a user interface. The shutdown of the circulatory system monitor 12 may facilitate saving power for later uses of the circulatory system monitor 12 and/or to prolong the life of the components of the circulatory system monitor 12 (e.g., to prevent a motor of the pump from burning out due to overuse, etc.).

If a best-positioned sensor assembly 10 meeting a baseline signal measurement requirement is identified at step 108, the circulatory system monitor 12 may use a signal from the best-positioned sensor assembly 10 during measurements (e.g., current and/or subsequent measurements) of the circulatory system parameter at the target area. For example, the circulatory system monitor 12 may use the signal from the identified or determined best-positioned sensor assembly 10 to compute 116 a systolic pressure and compute 118 a diastolic pressure. In some cases, once the best-positioned sensor assembly 10 meeting a baseline signal measurement requirement is determined, the other sensor assemblies 10 of the sensor array 14 may be turned off, however, this is not required. In some cases, it may be useful to use more than one sensor assembly 10 but less than all when determining one or more circulatory system parameters. For example, two of the sensor assemblies 10 may both return good signals, in which cases both signals may be processes and an average may be taken to identify the circulatory system parameter. In another example, two of the sensor assemblies 10 that are both determined to be in registration with the same artery, one upstream of the other, signals from both sensors may be used to determine a time of flight (e.g. velocity) of a heart beat pressure pulse through the artery. These are just examples.

Once the systolic pressure and the diastolic pressures are computed 116, 118, the circulatory system monitor 12 may display 120 the computed values on a display (e.g., the display 22 or other display) on a user interface (e.g., the user interface 20 or other user interface). The circulatory system monitor 12 may further determine 122 if the computed systolic and diastolic pressures are within a desired or preset range. If the computed pressures are not within a desired or preset range of values, the circulatory system monitor 12 may alert a user (e.g., the patient, a medical practitioner, and/or other user) to the computed pressures that are not within a desire range and send 126 the computed pressures to one or more remote servers (e.g., the remote server 28 or other remote servers) and/or one or more monitoring devices (e.g., a monitoring interface 26) for storage and possible further analyses. If it is determined 122 the values are within a desired or present range, the circulatory system monitor 12 may send the computed pressures to one or more remote servers and/or one or more monitoring devices for storage and possible further analyses. A controller (e.g., the processor or controller 16 or other controller) of the circulatory system monitor 12 may implement the approach 100 in an automated manner with or without manual or electronic prompting from a user.

The circulatory system monitor 12 may re-calibrate from time-to-time, periodically, whenever a value of the signal from the current best-positioned sensor assembly 10 changes by a predetermined amount, or at any other suitable time. In some cases, the circulatory system monitor 12 may re-calibrate (e.g. determine which pressure/force sensor of the pressure/force sensor array 14 is providing a strongest measure of the blood pressure at the target area based on the measured parameters) at each interval for computing systolic pressure and diastolic pressure. The intervals for computing systolic pressure and diastolic pressure may be preset automatically, preset by user entry, manually entered by a user, and/or set in one or more other manners.

In some cases, the circulatory system monitor 12 may be configured to determine a pulse and/or pulse wave velocity in the artery, which may be useful in detecting the onset of cardiovascular disease and/or cardiovascular risks of the patient. When detecting a pulse wave velocity, the circulatory system monitor 12 may, for example, determine a first sensor assembly (e.g., a first sensor assembly 10 or other first sensor assembly) and a second sensor assembly (e.g., a second sensor assembly 10 or other second sensor assembly) are both in good contact with a desired artery or other vessel, and then a controller and/or conditioning circuitry may utilize signals from the first sensor assembly 10 and the second sensor assembly 10, along with a timer and a known distance between the first sensor assembly 10 and the second sensor assembly 10, to determine a measure of the pulse wave velocity in an artery of the patient (i.e., a speed at which a pulse is traveling through the artery). A controller (e.g., the controller 16 or other controller) of the circulatory system monitor 12 may implement the approach for determining a pulse wave velocity in an automated manner with or without manual or electronic prompting from a user.

Having thus described several illustrative embodiments of the present disclosure, those of skill in the art will readily appreciate that yet other embodiments may be made and used within the scope of the claims hereto attached. It will be understood that this disclosure is, in many respects, only illustrative. Changes may be made in details, particularly in matters of shape, size, and arrangement of parts without exceeding the scope of the disclosure. The disclosure's scope is, of course, defined in the language in which the appended claims are expressed

What is claimed is:

1. A circulatory sensor monitor, comprising:
a force sensor array having a plurality of force sensors;
a controller in communication with the force sensor array; and
wherein with the force sensor array pressed against a patient's body, the controller is configured to identify a force sensor of the plurality of force sensors from which a signal is to be used to determine a circulatory system parameter of the patient's body and determine the circulatory system parameter of the patient's body based on the signal from the identified force sensor.

2. The circulatory sensor monitor of claim 1, further comprising:
an inflatable member in mechanical communication with the force sensor array, the inflatable member configured to apply a force to the force sensor array against the patient's body when inflated;
a pump in communication with the inflatable member for inflating the inflatable member.

3. The circulatory sensor monitor of claim 1, wherein the circulatory system parameter is blood pressure.

4. The circulatory sensor monitor of claim 1, wherein the controller is configured to run a calibration procedure to determine which force sensor of the force sensor array is in a best position to sense the circulatory system parameter of the patient's body.

5. The circulatory sensor monitor of claim 4, wherein the controller is configured to effect the calibration procedure by:
receiving signals from each force sensor of the force sensor array; and
determining which force sensor of the force sensor array is providing a strongest signal relative to the circulatory system parameter based on the received signals from each force sensor of the force sensor array.

6. The circulatory sensor monitor of claim 5, wherein the controller is configured to use the force sensor of the force sensor array that is providing the strongest signal relative to the circulatory system parameter for at least one subsequent determinations of the circulatory system parameter.

7. The circulatory sensor monitor of claim 6, wherein the controller is configured to turn off those force sensors of the force sensor array that are not providing the strongest signal relative to the circulatory system parameter.

8. The circulatory sensor monitor of claim 1, wherein:
the force sensor array has a first force sensor and a second force sensor; and
the controller is configured to use signals from the first force sensor and signals from the second force sensor to determine a pulse wave velocity in an artery of the patient's body.

9. The circulatory sensor monitor of claim 1, wherein:
the force sensor array has a first set of force sensors and a second set of force sensors;
the first set of force sensors are configured to output signals to a first sensor input of an application-specific integrated circuit (ASIC); and
the second set of force sensors are configured to output signals to a second sensor input of the ASIC.

10. The circulatory sensor monitor of claim 1, further comprising:
a display in communication with the controller, the controller is configured to provide information related to the circulatory system parameter to the display for displaying thereon.

11. The circulatory sensor monitor of claim 1, further comprising:
a communications component in communication with the controller, the communications component is configured to communicate information related to the signals from the force sensor array to a remote computing device.

12. A force sensor assembly, comprising:
a substrate;
a sense die having a diaphragm, the sense die secured relative to the substrate;
a cover secured relative to the substrate, the cover having an opening that is in registration with the diaphragm when the cover is secured relative to the substrate;
a force transfer mechanism extending at least partially into the opening of the cover and is in contact with the diaphragm of the sense die;
a film secured to the cover over the opening and in contact with the force transfer mechanism; and
wherein the force transfer mechanism is configured to transfer force applied to the film to the diaphragm of the sense die.

13. The force sensor assembly of claim 12, wherein the film comprises a nylon film.

14. The force sensor assembly of claim 12, wherein the film has a thickness between 0.1 millimeters (mm) and 0.2 mm.

15. The force sensor assembly of claim 12, wherein the film is laser welded to the cover.

16. The force sensor assembly of claim 12, wherein the force transfer mechanism comprises a ball bearing.

17. The force sensor assembly of claim 12, wherein the force transfer mechanism comprises a non-compressible fluid or gel contained in a defined cavity defined at least in part by the film.

18. The force sensor assembly of claim 12, wherein:
the sense die is one of a plurality of sense die secured relative to the substrate and forming a sense die array;
each sense die has a corresponding opening in the cover when the cover is secured relative to the substrate;
each opening has a corresponding force transferring mechanism extending at least partially into the corresponding opening; and
each opening in the cover has a corresponding film secured to the cover over the opening.

19. The force sensor assembly of claim 12, further comprising:
signal conditioning circuitry secured relative to the substrate and in communication with the sense die.

20. A method of using a force sensor assembly, the method comprising:
applying a contact force to a force sensor array having a plurality of force sensors against a target area of a patient, wherein each force sensor of the force sensor array is configured to provide a signal indicative of a corresponding force;
measuring a circulatory system parameter based on the signals from each force sensor in the force sensor array while the contact force is applied to the force sensor array against the target area of a patient;
determining which force sensor of the force sensor array is providing a strongest measure of the circulatory system parameter; and
using a signal from the force sensor of the force sensor array that is determined to be providing the strongest measure of the circulatory system parameter during subsequent measures of the circulatory system parameter at the target area.
